(19) United States
(12) Patent Application Publication
(10) Pub. No.: US 2009/0222048 A1
(43) Pub. Date: Sep. 3, 2009

(54) METHOD AND APPARATUS FOR BONE DISTRACTION PLATE

(76) Inventor: Darnell Kaigler, SR., Detroit, MI (US)

Correspondence Address: DICKSTEIN SHAPIRO LLP, 1825 EYE STREET NW, Washington, DC, 20006-5403, US

(21) Appl. No.: 12/394,480

(22) Filed: Feb. 27, 2009

Related U.S. Application Data
(60) Provisional application No. 61/064,377, filed on Feb. 29, 2008.

(51) Int. Cl. A61B 17/80

(52) U.S. Cl. 606/282

(57) ABSTRACT

The described embodiments relate to a bone distraction plate device, which is surgically implanted, for the purpose of growing new bone through the process of distraction. The device comprises a prosthesis housing a screw mechanism that attaches to a threaded post, which extends through tissue from an onlay plate and is surgically placed on the alveolar bone. After a brief, latent period, the screw mechanism is then activated daily until the desired amount of new bone growth (height and width) is achieved.

### CROSS REFERENCE TO RELATED APPLICATIONS

This application claims priority from U.S. Provisional Application No. 61/064,377, filed on Feb. 29, 2008, which is hereby incorporated by reference in its entirety.

### FIELD OF THE INVENTION

Embodiments described herein relate generally to dental implant systems and methods for growing new bone, more particularly, to dental implant systems and surgical methods for encouraging new bone growth in areas of the mouth that have suffered bone loss and most particularly to distraction dental implant devices and methods for forming new bone growth and soft tissue by distraction osteogenesis in areas of the jaw bone.

### BACKGROUND OF THE INVENTION

Orthopedic surgeons have conventionally relied upon the process of distraction osteogenesis to reconstruct and lengthen bones. This process may involve placing a vascularized piece of bone under tension, thereby inducing native bone formation via the creation of a bony reparative callus, which can then be placed under tension to generate new bone. To effect distraction osteogenesis, a surgeon generally performs an osteotomy where sectioning or segmenting the bone into more than one piece occurs. As the bone segments heal, they will gradually expand over a period of time; the gradual expansion allows the blood vessels and nerve ends to remain intact during the distraction process. For example, the bone may extend a millimeter a day, often by performing two extensions of half a millimeter, for three or four days which allow the blood vessels and nerve ends to remain intact.

As the gap between the bone segments widens, the natural healing capacity of the body can fill the void with new bone and adjacent soft tissue. Once the desired bone formation is achieved, the area may be allowed to heal and consolidate. Often, the distraction osteogenesis device is then removed.

Premature tooth loss has limited a patient's ability to chew and speak clearly. In response, a growing number of patients are requesting tooth replacement. Conventionally, dentists have been able to replace missing teeth by various means such as a removable prosthesis (partial or complete dentures). Dentists have also used the placement of fixed bridge work cemented to adjacent teeth as a solution. These two conventional methods serve to fill the void of the edentulous space by replacing the crown of the involved teeth; these methods, however, fail to cure other problems associated with premature tooth loss, e.g., bone deterioration.

Bone deterioration limits the surgical options available to dentists requiring a dentist to place a smaller than optimal sized dental implant. These smaller dental implants cannot accommodate the mechanical load from chewing and ultimately may loosen and/or fail. Moreover, the bone deterioration may cause a dental implant to be placed in a less than ideal location that is not as aesthetic or functional as would be considered optimal.

One prior solution to this bone deterioration problem (if the bone loss was not significant) was to augment the bony bed with the patient's own bone or cadaveric bone, e.g., grafting, as a transplant, or with synthetic bone substitutes. If the bone loss was significant, the bone augmentation must be done as a first surgical procedure with the placement of the dental implant occurring several months later, as a second surgical procedure (once healing of the bone graft was completed).

There is a need for a new distraction device and method for allowing the rapid regeneration of new bone growth, reducing a patient's aesthetic concerns, reducing the need for bone grafts, and preventing the actual cutting of the bone in an area of bone deficiency.

### BRIEF SUMMARY OF THE INVENTION

The described embodiments relate to a bone distraction plate device, which is surgically implanted, for promoting new bone growth through the process of distraction. A specific embodiment includes a device comprising of a prosthesis housing a screw mechanism that attaches to a threaded post, which extends through tissue (transmucosa) from an onlay plate (comprising materials such as metals, bio-ceramics, bio-polymers or any combination thereof), and is surgically placed on the alveolar bone. After a brief, latent period, the screw mechanism of the device is activated daily until the desired amount of new bone growth (height and width) is achieved.

### BRIEF DESCRIPTION OF THE DRAWINGS

FIGS. 1-3 illustrate an embodiment described herein.

FIG. 4 illustrates a second embodiment described herein.

FIG. 5 illustrates a third embodiment described herein.

FIGS. 6-9 illustrate various stages of operating the FIG. 1 embodiment.

FIG. 10 illustrates a fourth embodiment described herein.

### DETAILED DESCRIPTION OF THE INVENTION

Embodiments discussed herein provide techniques and apparatuses for promoting new bone growth and soft tissue by distraction osteogenesis in areas of the jaw bone and/or maxillofacial region. In the following description, numerous specific details are set forth, such as material types, dimensions, specific tissues, etc., in order to provide a thorough understanding of the invention. Practitioners having ordinary skill in the biomedical arts will understand that the invention may be practiced without many of these details. In other instances, well-known devices, methods, and biochemical processes have not been described in detail to avoid obscuring the invention.

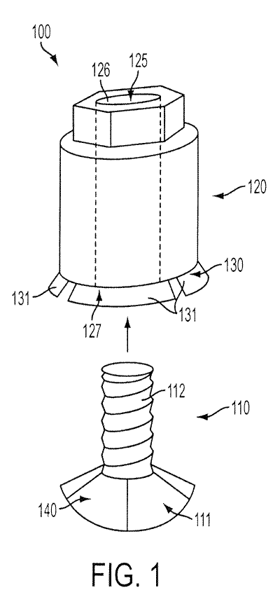

Embodiments discussed herein offer solutions to the foregoing problems by providing a bone distraction plate device that can enhance the structural integrity, and reduce bone deterioration. According to one embodiment (FIGS. 1-3), the bone distraction plate device 100 comprises a plate component 110 and an expansion component 120. The plate component 110 has a disc portion 111 and a threaded cylinder portion 112 (or apical portion) extending vertically from the disc center of the portion 110. The expansion component 120 (coronal portion) operatively connects and controls the retraction of the plate component 110. The plate component 110 and expansion component 120 can independently be formed of a material selected from one or more of the following materials: commercially pure Grade IV titanium, metal alloys or other metal substances. It should be noted that the metal substance should meet or exceed the parameters for materials used in dental implantology. It should be also appreciated that the plate and expansion components 110, 120 can be formed of a degradable or non-degradable bioceramic material, e.g., hydroxyapatite, reinforced polyethylene composite, betatricalciumphosphate, substituted calcium phosphates, bioactive glass, resorbable calcium phosphate, alumina, zirconia, etc. that may be manufactured as a solid structure. It should also be noted that a biodegradable polymer can be used in combination with the bioceramic material to form a composite material used to form the plate and expansion components 110, 120. In the preferred embodiment, a hydroxyapatite material is utilized to form the plate and expansion components 110, 120. The plate and expansion components 110, 120 can be formed by any type of material known in the art having characteristics that result in non-toxic byproducts.

For example, plate and expansion components 110, 120 can be formed of synthetic polymers (alone or in combination) such as polyurethanes, polyorthoesters, polyvinyl alcohol, polyamides, polycarbonates, poly(ethylene)glycol, polylactic acid, polyglycolic acid, polycaprolactone, polyvinyl pyrrolidone, marine adhesive proteins, and cyanoacrylates, or analogs, mixtures, combinations, and derivatives of the above. Plate and expansion components 110, 120 can also be formed of naturally occurring polymers or natively derived polymers (alone or in combination) such as agarose, alginate, fibrin, fibrinogen, fibronectin, collagen, gelatin, hyaluronic acid, and other suitable polymers and biopolymers, or analogs, mixtures, combinations, and derivatives of the above. Also, plate and expansion components 110, 120 can be formed from a mixture of naturally occurring biopolymers and synthetic polymers. Alternatively, plate and expansion components 110, 120 can be formed of a collagen gel, a polyvinyl alcohol sponge, a poly(D,L-lactide-co-glycolide) fiber matrix, a polyglactin fiber, a calcium alginate gel, a polyglycolic acid mesh, polyester (e.g., poly-(L-lactic acid) or a polyanhydride), a polysaccharide (e.g., alginate), polyphosphazene, or polyacrylate, or a polyethylene oxide-polypropylene glycol block copolymer. Plate and expansion components 110, 120 can be produced from proteins (e.g. extracellular matrix proteins such as fibrin, collagen, and fibronectin), polymers (e.g., polyvinylpyrrolidone), or hyaluronic acid. Synthetic polymers can also be used, including bioerodible polymers (e.g., poly(lactide), poly(glycolic acid), poly(lactide-co-glycolide), poly(caprolactone), polycarbonates, polyamides, polyanhydrides, polyamino acids, polyortho esters, polyacetals, polycyanoacrylates), degradable polyurethanes, non-erodible polymers (e.g., polyacrylates, ethylene-vinyl acetate polymers and other acyl substituted cellulose acetates and derivatives thereof), non-erodible polyurethanes, polystyrenes, polyvinyl chloride, polyvinyl fluoride, poly(vinylimidazole), chlorosulphonated polyolifins, polyethylene oxide, polyvinyl alcohol, teflon(R), and nylon.

Bioceramic materials employed as the manufacturing material can fall into all three biomaterial classifications, i.e., inert, resorbable and active, meaning they can either remain unchanged, dissolve or actively take part in physiological processes. There are several calcium phosphate ceramics that are considered biocompatible and possible materials for the plate component 110. Of these, most are resorbable and will dissolve when exposed to physiological environments, e.g., the extracellular matrix. Some of these materials include, in order of solubility: Tetracalcium Phosphate (Ca₄P₂O₉)>Amorphous calcium Phosphate>alpha-Tricalcium Phosphate (Ca₃(PO₄)₂)>beta-Tricalcium Phosphate (Ca₃(PO₄) 2)>>Hydroxyapatite (Ca₁₀(PO₄)₆(OH)₂). Unlike the other certain calcium phosphates listed above, hydroxyapatite does not break down under physiological conditions. In fact, it is thermodynamically stable at physiological pH and actively takes part in bone bonding, forming strong chemical bonds with surrounding bone. This property is advantageous for rapid bone repair after surgery. Other bioceramic materials such as Alumina and Zirconia are known for their general chemical inertness and hardness. These properties can be exploited for implant device support purposes, where it is used as an articulating surface for implant devices. Porous alumina can also be used as a bone spacer, where sections of bone have had to be removed due to various conditions or diseases. The material acts as an environment that promotes bone growth.

The outer surface of both the plate and expansion components 110, 120 (especially the plate component 110) can be conventionally covered/roughened with a surface coating for additional bone growth advantages known by one of ordinary skill in the art. The plate and expansion components 110, 120 having corresponding cylinder like portions (threaded cylinder portion 112 and hollow slot 125 (described below)), and can be conventionally threaded (externally on the plate component 110 and internally on the expansion component 120) with clockwise or counterclockwise treads. The threads of the plate component 110 start about two (2) mm (for example) from the base of the plate component 110 and continue vertically along the entire length of the cylinder 112 of the plate component 110.

The expansion component 120, on the other hand, has a hollow slot 125 extending completely through, and within the full length, of the expansion component (completely from the top end 126 to the bottom end 127 of the expansion component 120) having threads. The hollow slot 125 has a cylindrical configuration and comprises internal clockwise or counterclockwise threads that correspond to respective threads on the cylinder 112 of the plate component 110. The pitch of the threads on the plate and expansion components 110, 120 can be any pitch that promotes new bone growth of approximately 0.5 mm/day. Examples of a pitch that promotes new bone growth include, for example, 0.25 mm, 0.3 mm, 0.5 mm, 1.0 mm, 1.5 mm and 2.0 mm. The length of the expansion component 120 may vary depending on the required distraction; an example includes a length of the expansion component 120 of approximately 3.5 mm. In order to enable the surgeon or patient to easily read the distance of distraction after having activated the distraction expansion component 120 (as described below), the head of the expansion component 120 is preferably marked on the surface between the center and the side of the expansion component 120. The mark may be an indentation in the expansion component 120 and/or may consist of a different color.

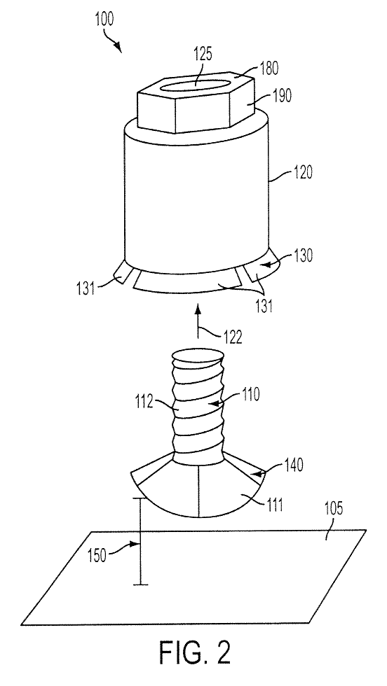

Prior to, during and after initial placement of the bone distraction plate device 100, the horizontal interface 122 between the plate and expansion components 110, 120 has a wide, noticeable gap 150 (See FIG. 2). This interface may be smooth or have interlacing or interlocking complimentary locking members 131 on the facing surfaces 130, 140 of the plate and expansion components 110, 120, respectively such members 13 1 would prevent rotation and torsion at the horizontal interface 122 during the healing process and before the plate and expansion components 110, 120 become integrated with the bone 105. As described below, the expansion component 120 of the bone distraction plate device 100 provides for retraction between the plate and expansion components 110, 120 to form a distraction gap 150, between the plate component 110 and the bone 105. (See FIGS. 1 and 2).

As illustrated in FIGS. 1 and 2, the expansion component 120 must be rotatable around the plate component 110, as will be discussed in detail below. As mentioned above, expansion component 120 has internal threads that can operatively engage with external threads of plate component 110 of the bone distraction plate device 100 during implantation. The expansion component 120 is rotated and thus must not be fixedly connected to the plate component 110 in such a way as to prevent the expansion component 120 from freely rotating around the plate component 110 as the plate component 110 rotationally raises from the bone 105 as the gap 150 between the plate and expansion components 110, 120 is decreased axially during implantation by the interaction of the internal threads of the expansion component 120 with the external threads of the plate component 110. Other conventional means for maintaining the rotatability of the expansion component 120 would be acceptable. The plate and expansion components 110, 120 may also be used to carry the load of the bone distraction plate device 100 by applying vertical forces to the alveolar bone.

The plate component 110 remains stationary in the bone and rotational movement of the expansion component 120, provided by, such as for example, the interaction of the threads of the expansion component 120 with the external threads of the plate component 110, provide for the retraction of the plate component 110 to the expansion component 120 (See FIGS. 6-9). The body then attempts to heal itself by filling in the gap 150 with new bone 106. If the gap 150 is widened daily, the body recognizes the newly expanded gap 150 and continues to fill the gap 150 with new bone 106. By expanding the gap 150 slowly over time (0.5-2.0 millimeters per day), the body will continue to heal the gap 150 and generate new bone 106. Consequently, because the native bone is utilized as the template for repair, the new bone 106 generated will comprise the same size and shape as the original bone. Such results are advantageous and unique to new bone generation and are not accomplished when using other conventional bone transplantation techniques. Furthermore, during distraction osteogenesis, in addition to creating new bone, the overlying soft tissues are regenerated, a secondary gain unique to distraction osteogenesis. This secondary beneficial effect has significant clinical implications, for not only is the underlying foundation properly established, but also the overlying soft tissue is recreated providing for aesthetic and functional rehabilitation of the defect.

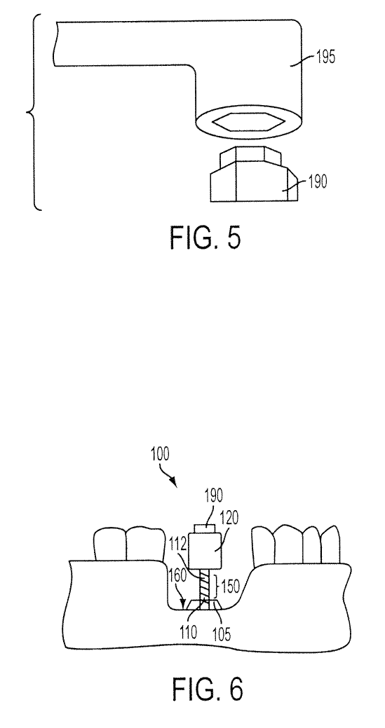

When seated, the plate component 110 sits flush with the surface 160 of the bone 105, while the threaded cylinder 112 extends beyond the surface of the plate component 110 through the mucosa 170, intraorally (See FIG. 6). Initially during implementation, the expansion component 120 sits atop the threaded cylinder 112. The top surface 180 of the expansion component 120 has a hexagonal shaped aperture 190. The aperture 190 provides the mechanical access to rotate the expansion component 120 to activate the distraction process via a corresponding L or T shaped hexagonal key 195 (See FIG. 5). The hexagonal key is made from stainless steel, and causes retraction of the plate and expansion components 110, 120 of the bone distraction plate device 100 during operation, as will be described more fully below.

At times, biodegradable polymers suffer from warping, hollowing or substantial erosion inherent with the process of degradation. In order to manage such a problem, polymers with high crystallinity are utilized. Self-reinforced and ultrahigh strength bioabsorbable composites are readily assembled from partially crystalline bioabsorbable polymers, like polyglycolides, polylactides and glycolide/lactide copolymers. These materials have high initial strength, appropriate modulus and strength retention time from 4 weeks up to 1 year in-vivo, depending on the implant geometry. Reinforcing elements such as fibers of crystalline polymers, fibers of carbon in polymeric resins, and particulate fillers, e.g., hydroxyapatite, may also be used to improve the dimensional stability and mechanical properties of biodegradable devices. The use of interpenetrating networks (IPN) in biodegradable material construction has been demonstrated as a means to improve mechanical strength. To further improve the mechanical properties of IPN-reinforced biodegradable materials, biodegradable plates may be prepared as semi-interpenetrating networks (SIPN) of crosslinked polypropylene fumarate within a host matrix of poly(lactide-co-glycolide) 85:15 (PLGA) or poly(l-lactide-co-d,l-lactide) 70:30 (PLA) using different crosslinking agents.

Resin composites with incorporated polytetrafluoroethylene (PTFE) particles improve the hydrophobicity and surface properties of device implants, e.g., components 110, 120. PTFE has high resistance to chemical regents, low surface energy, tolerance to low and high temperatures, resistance to weathering, low friction wiring, electrical insulation, and slipperiness. However, because conventional PTFE has poor resistance to abrasion, the inventor contemplates cross-linking PTFE with gamma-beam irradiation can be employed to drastically enhances resistance to abrasion and deformation. Further, the composites made of braided carbon fibers and epoxy resins (so called biocompatible carbon-epoxy resin) have better mechanical properties than composites made of short or laminated unidirectional fibers.

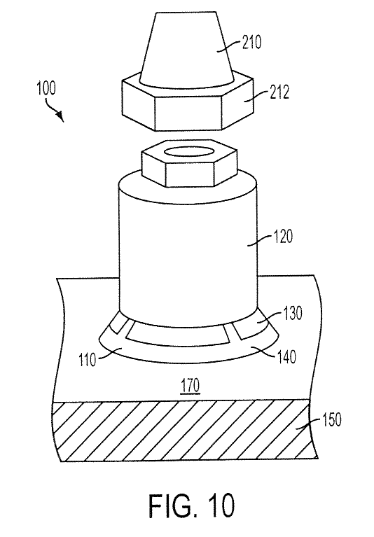

FIG. 10 illustrates a second embodiment in which an abutment component 210 can be integrated with the bone for attaching a prosthesis such as, for example, a crown, a bridge or dentures. The abutment component 210 has essentially the same dimensions of the expansion component 120. The abutment component 210 is the component utilized as the base for a prosthesis such as, for example, a crown, a bridge or dentures after sufficient bone growth through distraction osteogenesis has been generated by the controlled separation of the two components 110, 120. The end 212 of the abutment 210 must be operative to interlock with the expansion component 120 such that during bone integration, the two components 110, 120 remain fixedly connected.

As illustrated in FIGS. 4-9, the operation of the bone distraction plate device 100 is described. The bone distraction plate device 100 is placed atop a series of aerated holes 198 formed within the alveolar bone in a predetermined site or area 197 where additional bone is required. Prior to any surgical technique, proper treatment planning should be performed, including a physical examination, X-ray studies and consultation with the dentist fabricating any necessary prosthesis.

Once the patient has been conventionally prepared for surgery, a local anesthetic is given and infiltrated into the surgical site. After allowing adequate time for anesthesia and vasoconstriction, aerated holes 198 are made along the crest of dental ridge, in the predetermined site. The underlying bone is conventionally exposed by raising a full thickness mucoperiosteal flap with an elevator. The exposed bone is conventionally evaluated by palpitation for bone density and quality.

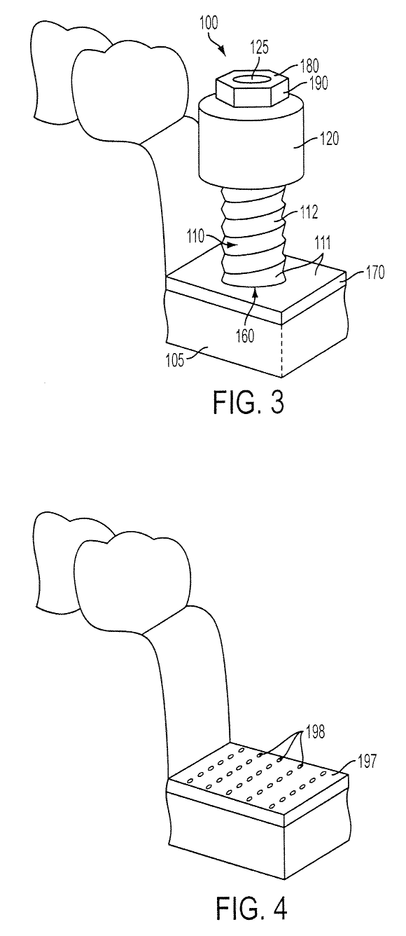

In other embodiments using conventional drill methods, an osteotomy is created in the planned implant placement site. It should be noted that other conventional procedures could be used to create the osteotomy. All of the bone drilling procedures include copious amounts of irrigation, (internally and/or externally). The osteotomy site is enlarged by utilizing progressively wider drills. Optionally, the parallelism of the osteotomy site can be verified by X-rays. The final sized osteotomy site is completed by either utilizing the final, smooth, twist drill or by tapping in the threads corresponding to the combination distraction dental implant. At this point, the distraction plate device 100 is placed (See FIG. 3) onto or into the bone 105 manually or by use of a conventional implant drill set at slow speeds, as is known by those skilled in the art. The wound is irrigated and, if osteotomies are formed, the incisions are conventionally closed with the aperture 190 being exposed, as described above. Osseous integration of the distraction plate device 100 into the bone is verified visually and tactilely. At this point, the patient has been prepared for the process of growing new bone by distraction osteogenesis prior to any prosthesis such as, for example, a crown, a bridge or dentures, if any, being connected.

The patient is then educated as to the care and activation of the distraction plate device 100. After allowing for a period of initial healing, a latency period (of about 5-7 days), the adjustable expansion component 120 is activated or maneuvered, (turned) thereby retracting the plate component 110 to the expansion component 120 (about 1.0 mm per day) in divided doses, and thus creating a distraction gap 150 above the bone. The patient is also educated to make the adjustment necessary to increase or widen the gap 150 each day. Thereafter, the patient is seen for follow-up and evaluation as appropriate. Since the typical height of a natural tooth crown above the gum is about eight (8) mm, in order to properly function, the distal end 185 of the expansion component 120 should not extend above the level of the lowest adjacent tooth crown.

After sufficient bone height (about 5 mm to about 15 mm) is achieved, the distraction process is halted and the expansion component 120 is removed and replaced with a prosthesis. It should be appreciated, however, that the expansion component 120, in some embodiments, can be left in place. The newly grown bone 106, however, is still relatively weak and incompletely ossified, a period of about four to about six weeks is required before the fabrication and installation of the final prosthesis such as, for example, a crown, a bridge or dentures. Additionally during this period, a prosthesis, e.g., abutment component 210, can replace the expansion component 120, or be used in combination with the expansion component 120 (e.g., abutment component 210), and becomes incorporated with the bone thereby increasing the rigidity of the installed bone distraction plate device 100.

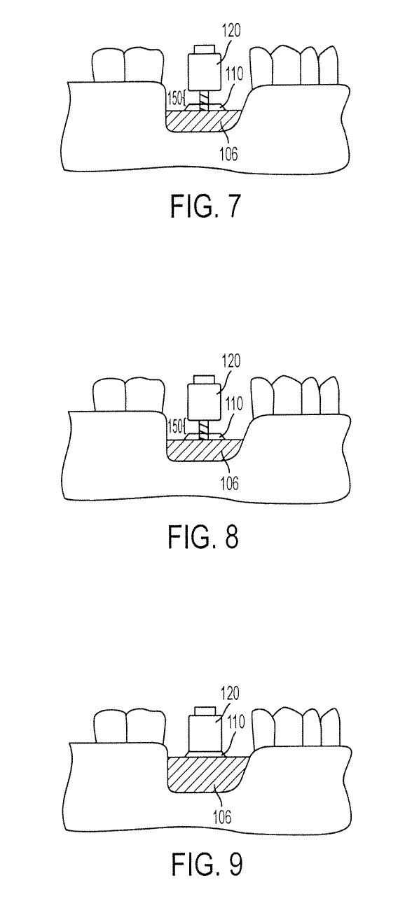

FIGS. 6-9 illustrates the distraction process according to an embodiment described herein. FIG. 6 shows the bone distraction plate device 100 newly installed in an area having insufficient bone to support an optimal dental implant. FIG. 7 shows an intermediate bone growth situation where some bone has been regenerated but not enough to support an optimal dental implant. It should be noted that the separation between the plate 110 and expansion 120 components has decreased, i.e., a decreased distraction gap 150. FIG. 8 shows another intermediate bone regeneration position. FIG. 9 shows the final result if an abutment is positioned on the distraction plate device 100.

The foregoing description illustrated one specific application of the technique and technology of distraction osteogenesis to the field of dental implants using an exemplary distraction plate device and method. Since conventional dental implants have similar basic forms, it should be apparent to those skilled in the art that the potential combinations of the distraction plate device is unlimited. By modifying minor details of the basic design, such as, for example, splitting the dental implant horizontally 60/40% rather than the 50/50% as described, altering the length or taper of the expansion component, changing the pitch of the screws, etc., are just a few of the unlimited possible variations.

Nevertheless, in all possible variations, the basic concept remains as described, i.e., utilizing the bone distraction plate device 100 having plate and expansion components 110, 120 to achieve sufficient bone generation in an area of deficient bone in order to place an optimum dental implant. Advantages of embodiments described herein include providing new bone growth and soft tissue formation, thereby, reducing the number and morbidity of surgical procedures a patient is subjected to during the distraction as compared to the prior surgical procedures. Additionally, the distraction plate device described above provides for increased versatility by using an expansion component 120 to continuously adjust the distraction gap 150 during the bone regeneration process without additional surgical procedures.

One way to enhance the bone healing process during this procedure would be to introduce bone growth factors such as bone morphogenetic proteins (BMPs) and basic fibroblast growth factor (bFGF) to the area of distraction. These two classes of bone growth factors have been shown to accelerate bone regeneration, bone healing to prosthetic-like implants, and increase strength and stability to the bony callus. The bone growth factors could be delivered to the area of distraction by a variety of methods. One method would be to introduce the bone growth factors in combination with a collagen matrix, which could be a gel- or sponge-like material, to the area of distraction. The bone growth factor would stimulate the patient's own bone cells into action, while the collagen would provide the scaffolding into which the stimulated bone cells can grow. In the end, bone could replace the collagen scaffold, which may be eventually resorbed. Fibrinogen, a-thrombin, as well as other various antibiotics, growth hormones, gene therapies, or combinations of these factors may also be utilized in the distraction plate device 100 to promote healthy bone growth. The BMP material may be infused as a liquid or viscous gel substance.

Another method of delivery could be to coat the actual bone distraction plate device 100 with the bone growth factor in combination with hydroxyapatite, which would have a synergic stimulative effect on the bone cells. For this to be accomplished, a specific amount of the bone growth factor would be absorbed to a gritblasted hydroxyapatite coated implant or distraction plate device prior to implantation.

The embodiments of the bone distraction plate device and method reduce the number of surgical procedures required to place a dental implant in an area initially having insufficient bone to support an optimal implant and is more aesthetically pleasing during the actual distraction process as compared to conventional devices and methods. It should be also be appreciated to those skilled in the art that the above concept of a bone distraction plate device is not limited to use as a dental implant and could be used as a general distraction device in the maxillofacial region.

Changes and modifications in the specifically described embodiments and methods can be carried out without departing from the scope of the invention which is intended to be limited only by the scope of the appended claims.

What is claimed as new and desired to be protected by Letters Patent of the United States is:

1. A bone distraction device comprising:
a plate component for growing new bone in an area of deficient bone; and
an expansion component for operatively connecting and controllably retracting the plate component.

2. The bone distraction device of claim 1, wherein said plate component is a disc configuration with a threaded cylinder extending vertically from its center.

3. The bone distraction device of claim 1, wherein said expansion component comprises a hollow slot for retracting the plate component.

4. The bone distraction device of claim 3, wherein said hollow slot has a cylindrical configuration and comprises internal clockwise threads.

5. The bone distraction device of claim 1, wherein said plate and expansion components comprise hydroxyapatite material.

6. The bone distraction device of claim 1, wherein said plate component comprises a bioceramic material.

7. The bone distraction device of claim 1, wherein said expansion component comprises a bioceramic material.

8. The bone distraction device of claim 1, wherein the expansion component is rotatable around said plate component.

9. The bone distraction device of claim 1 further comprising an abutment for attaching a prostheses.

10. The bone distraction device of claim 1, wherein said plate and expansion components are used intraorally.

11. A method of rapid bone regeneration comprising:
adjusting an expansion component;
by adjusting said expansion component, retracting a plate component from a surface of a bone;
creating a gap between said plate component and said surface of the bone; and
by creating this gap, allowing new bone to grow to achieve bone generation in an area of deficient bone.

12. The method of claim 11, further comprising, after sufficient bone growth has occurred, removing said expansion component.

13. The method of claim 12, further comprising replacing said removed expansion component with an abutment for a prosthesis.

14. The method of claim 11, wherein said method is used intraorally.

15. A method of manufacturing a bone distraction device comprising:
forming a plate component for growing new bone in an area of deficient bone; and
forming an expansion component for operatively connecting and controllably retracting the plate component.

16. The method of claim 15, wherein said plate forming step comprises forming said plate component as a disc configuration with a threaded cylinder extending vertically from its center.

17. The method of claim 16, wherein said plate forming step comprises forming said plate component with hydroxyapatite material.

18. The method of claim 15, wherein said plate forming step comprise forming said plate component with a bioceramic material.

19. The method of claim 15, wherein said expansion forming step comprises forming said expansion component with a hollow slot for retracting the plate component, said hollow slot having a cylindrical configuration and comprising internal clockwise threads within said hollow slot.

20. A bone distraction plate comprising:
a disc-shaped component for growing new bone in an area of deficient bone, said disc-shaped component having a threaded cylinder screw-mechanism extending vertically from its center.

21. The bone distraction plate of claim 20, wherein said disc-shaped component is surgically inserted intraorally.

22. A bone distraction plate comprising:
a plate component having an extension mechanism for extending said plate component from a surface of a bone, wherein said plate component is for growing new bone in an area of deficient bone.

23. The bone distraction plate of claim 22, wherein said plate component is employed inside of a patient's mouth.
